(19) United States
(12) Patent Application Publication
(10) Pub. No.: US 2014/0045951 A1
(43) Pub. Date: Feb. 13, 2014

(54) SYNTHETIC VITREOUS MATERIAL

(71) Applicants: Koji Uesugi, Kasugai-shi (JP); Ryuhei Hayashi, Suita-shi (JP); Yasutaka Hayashida, Suita-shi (JP); Koichi Baba, Suita-shi (JP); Kohji Nishida, Suita-shi (JP)

(72) Inventors: Koji Uesugi, Kasugai-shi (JP); Ryuhei Hayashi, Suita-shi (JP); Yasutaka Hayashida, Suita-shi (JP); Koichi Baba, Suita-shi (JP); Kohji Nishida, Suita-shi (JP)

(73) Assignees: OSAKA UNIVERSITY, Suita-shi (JP); MENICON CO., LTD., Nagoya-shi (JP)

(21) Appl. No.: 14/114,102

(22) PCT Filed: Apr. 6, 2012
(86) PCT No.: PCT/JP2012/059498

§ 371 Date: Oct. 25, 2013

(30) Foreign Application Priority Data
Apr. 27, 2011 (JP) 2011-099564

(51) Int. Cl. A61K 9/00; A61K 47/42

(52) U.S. Cl. CPC A61K 9/0051; A61K 47/42
USPC 514/773

(57) ABSTRACT

A synthetic vitreous material contains a self-assembling peptide and a salt, and has an osmotic pressure of 40 mOsm/kg to 200 mOsm/kg. Such a synthetic vitreous material is not toxic to ocular tissue, is capable of maintaining an intraocular tamponade effect over a long term, and is easy to handle and inject into an eye of a patient.

### TECHNICAL FIELD

The present invention relates to a synthetic vitreous material.

### BACKGROUND ART

A vitreous body is a gel-like substance which is comprised of collagen and sodium hyaluronate and fills the posterior cavity of an eyeball. The vitreous body has a tamponade effect by which a retina is pressed from inside of an eyeball, thereby preventing detachment of the retina. Thus, the vitreous body is an important tissue for the eyeball to function normally. At present, vitrectomy is often utilized for vitreoretinal disease which requires operation and treatment; however, the removed vitreous body is not replaced with any material because no suitable substitute for a vitreous body is available. Further, it is reported that, after a vitreous body is removed, the effect of intraocular drug administration via an injection diminishes in comparison to that in the presence of a vitreous body.

Examples of a tamponade material to be used after the removal of a vitreous body include gases (for example, air or an expansion gas such as sulfur hexafluoride or octafluoropropane), silicone oils, and perfluorocarbon liquids. Of those, silicone oil is frequently used clinically. However, silicone oil is highly toxic to the ocular tissue, and hence needs to be removed after a certain period of time, thus requiring troublesome handling. In addition, silicone oil may, for example, emulsify and become cloudy. Further, perfluorocarbon liquid is used as a temporary tamponade material during operation. However, perfluorocarbon liquid is also highly toxic to the ocular tissue and thus is removed from the eye when the eye operation has been completed. When gas tamponade is performed by using air or an expansion gas such as sulfur hexafluoride or octafluoropropane, such a gas is absorbed intraocularly, and hence the effect of the gas tamponade is maintained for only a short period of time of one day to one week (for example, Patent Literature 1). Further, when such gas tamponade is performed during operation of a patient, the patient usually is forced to lie in a prone posture for about one week after the operation. Collagen and hyaluronic acid, and salts thereof, are also used as tamponade materials (for example, Patent Literature 2). However, they are derived from organisms and/or microorganisms, are thus expensive, and hence have not yet been used in clinical applications.

Further, a composition using a polyethylene glycol, one of the ends of which is modified with a long-chain alkyl group, and a polyethylene glycol, both ends of which are modified with long-chain alkyl groups, have been proposed as tamponade materials (for example, Patent Literature 3). This composition has a high hardness and can be expected to exert a tamponade effect, but it is necessary to use a thicker injection needle (21 gauge) than a commonly-used injection needle (25 gauge) in order to improve their handleability. Thus, a larger load is applied to the eyeball and the period of time over which treatment is given may be longer.

### CITATION LIST

### Patent Literature

Patent Literature 1: JP 6-154263 A
Patent Literature 2: JP 5-184663 A
Patent Literature 3: JP 2010-104632 A

### SUMMARY OF THE INVENTION

### Problem(s) to be Solved by the Invention

The present invention has been made to solve the above-mentioned problems, and an object of the present invention is to provide a synthetic vitreous material which is not toxic to the ocular tissue, is capable of maintaining an intraocular tamponade effect over a long term, and has excellent handleability.

### Means for Solving the Problem(s)

A synthetic vitreous material is provided according to the present invention. The synthetic vitreous material includes a self-assembling peptide and a salt, and has an osmotic pressure of 40 mOsm/kg to 200 mOsm/kg.

In a preferred embodiment, the above-mentioned synthetic vitreous material includes the above-mentioned self-assembling peptide at 0.01 w/v % to 0.5 w/v %.

In a preferred embodiment, the above-mentioned self-assembling peptide is represented by the following amino acid sequence.

| | |
|---|---|
| Amino acid sequence: | a₁b₁c₁b₂a₂b₃db₄a₃b₅c₂b₆a₄ |

(In the amino acid sequence, a₁ to a₄ each represent a basic amino acid residue, b₁ to b₆ each represent a non-charged polar amino acid residue and/or a hydrophobic amino acid residue, provided that at least five amino acid residues out of the b₁ to b₆ are each a hydrophobic amino acid residue, c₁ and c₂ each represent an acidic amino acid residue, and d represents a hydrophobic amino acid residue.)

In a preferred embodiment, b₁ to b₆ in the above-mentioned amino acid sequence each independently represent an alanine residue, a valine residue, a leucine residue, or an isoleucine residue.

In a preferred embodiment, d in the above-mentioned amino acid sequence represents an alanine residue, a valine residue, a leucine residue, or an isoleucine residue.

### Effects of the Invention

The synthetic vitreous material of the present invention is capable of maintaining its intraocular tamponade effect over a long term. Further, the synthetic vitreous material of the present invention has moderate fluidity, thus having excellent handleability. The synthetic vitreous material of the present invention includes a self-assembling peptide and a salt, and is not toxic to the ocular tissue. Thus, when the synthetic vitreous material of the present invention is used, it is not necessary to maintain such a prone state after operation, as was required after gas tamponade, or to perform such a removal operation, as was required when silicone oil or the like is used; hence the quality of life (QOL) of the patient can be improved. Further, the synthetic vitreous material of the present invention also exhibits an excellent drug retention capability, thus being able to prevent the reduction of the effects of intraocular drug administration after the removal of a vitreous body.

### BRIEF DESCRIPTION OF THE DRAWINGS

FIG. 1a is a photograph of an anterior eye segment of a domestic rabbit, the photographbeing taken one week after injection of a synthetic vitreous material of the present invention.

FIG. 1b is a photograph of an ocular fundus of a domestic rabbit, the photograph being taken one week after injection of the synthetic vitreous material of the present invention.

FIG. 1c is a photograph of an HE-stained retinal tissue of a domestic rabbit, the photograph being taken one week after injection of the synthetic vitreous material of the present invention.

FIG. 2a is a photograph of an anterior eye segment of a domestic rabbit, the photograph being taken one month after injection of the synthetic vitreous material of the present invention.

FIG. 2b is a photograph of an ocular fundus of a domestic rabbit, the photograph being taken one month after injection of the synthetic vitreous material of the present invention.

FIG. 2c is a photograph of an HE-stained retinal tissue of a domestic rabbit, the photograph being taken one month after injection of the synthetic vitreous material of the present invention.

FIG. 3a is a photograph of an anterior eye segment of a domestic rabbit, the photograph being taken three months later after injection of the synthetic vitreous material of the present invention.

FIG. 3b is a photograph of an ocular fundus of a domestic rabbit, the photograph being taken three months after injection of the synthetic vitreous material of the present invention.

FIG. 3c is a photograph of an HE-stained retinal tissue of a domestic rabbit, the photograph being taken three months after injection of the synthetic vitreous material of the present invention.

### MODE(S) FOR CARRYING OUT THE INVENTION

<Definitions of Terms>

(1) Herein, the term “self-assembling peptide” refers to peptides which spontaneously associate in a solvent via interactions between peptide molecules. The interactions are not particularly limited, and examples thereof include hydrogen bonding, interionic interactions, electrostatic interactions such as the van der Waals force, and hydrophobic interactions. In one embodiment, self-assembling peptides are capable of self-assembling into a nanofiber or a gel in a room-temperature aqueous solution (such as an aqueous solution including the peptide at 0.4 w/v %).
(2) Herein, the term “gel” refers to a viscoelastic substance having both a viscous property and an elastic property.
(3) Herein, the term “hydrophilic amino acid” is meant to include: basic amino acids such as arginine (Arg/R), lysine (Lys/K), and histidine (His/H); acidic amino acids such as aspartic acid (Asp/D) and glutamic acid (Glu/E); and non-charged polar amino acids such as tyrosine (Tyr/Y), serine (Ser/S), threonine (Thr/T), asparagine (Asn/N), glutamine (Gln/Q), and cysteine (Cys/C). The letters in parentheses respectively refer to the three letter code and the one letter code of the amino acid.
(4) Herein, the term “hydrophobic amino acid” is meant to include nonpolar amino acids such as alanine (Ala/A), leucine (Leu/L), isoleucine (Ile/I), valine (Val/V), methionine (Met/M), phenylalanine (Phe/F), tryptophan (Trp/W), glycine (Gly/G), and proline (Pro/P). The letters in parentheses respectively refer to the three letter code and the one letter code of the amino acid.

### <Synthetic Vitreous Material>

A synthetic vitreous material of the present invention include the self-assembling peptide and a salt. Because the synthetic vitreous material of the present invention includes the self-assembling peptide and a salt, the synthetic vitreous material is capable of maintaining a long-term, intraocular tamponade effect and has excellent handleability.

The synthetic vitreous material of the present invention has an osmotic pressure of 40 mOsm/kg to 200 mOsm/kg. Because the synthetic vitreous material has an osmotic pressure in the above-mentioned range, the synthetic vitreous material is capable of maintaining a longer-term, intraocular tamponade effect and can have excellent handleability. If a synthetic vitreous material has an osmotic pressure of more than 200 mOsm/kg, the synthetic vitreous material may have reduced transparency. Note that the osmotic pressure of a synthetic vitreous material can be measured by using osmometry (osmolality measurement method) in which a cryoscopic method is used and which is in accordance with the Pharmacopeia of Japan.

It is preferred that the pH of the synthetic vitreous material of the present invention is adjusted to physiological conditions (a pH of about 7.4), and the pH can be adjusted by using, for example, any suitable pH adjuster or buffer.

A. Salt

A salt similar to one contained in bodily fluids (for example, an aqueous humor) is preferred as the above-mentioned salt, and any suitable salt can be used. Examples of the above-mentioned salt include ionic salts such as sodium chloride and magnesium chloride. These salts may be used alone or in combination.

A salt in the form of a salt solution prepared by dissolving any suitable salt in any suitable solvent may be used as the above-mentioned salt. Examples of the solvent used for preparing the salt solution include distilled water. A commercially available salt solution may be used as the salt solution. Specific examples thereof include a physiological saline, a Ringer's solution, and a diluent for an intraocular irrigating solution such as an oxiglutatione solution (for example, a diluent for an oxiglutatione solution packed in BSS PLUS, which is a trade name and manufactured by ALCON JAPAN LTD., and a diluent for an oxiglutatione solution packed in Opeaqua (trademark), which is a trade name and manufactured by Showa Yakuhin Kako Co., Ltd.). These salt solutions may be used alone or in combination.

The proportion of the salt contained in the synthetic vitreous material of the present invention can be adjusted so that the synthetic vitreous material to be obtained has an osmotic pressure of 40 mOsm/kg to 200 mOsm/kg.

B. Self-Assembling Peptide

The self-assembling peptide to be used in the present invention is only required to be non-toxic to the biological body, in particular the ocular tissue, and any suitable self-assembling peptide may be used. The synthetic vitreous material of the present invention includes the self-assembling peptide at preferably 0.01 w/v % to 0.5 w/v %, more preferably 0.05 w/v % to 0.4 w/v %. Because the synthetic vitreous material includes the self-assembling peptide at a ratio within any of the above-mentioned ranges, the synthetic vitreous material is capable of maintaining its intraocular tamponade effect over a long period of time and can have excellent handleability. Attention has also been paid to self-assembling peptides usable as substrates for a drug delivery system. Thus, after the synthetic vitreous material of the present invention is injectedintraocularly, the synthetic vitreous material can prevent the reduction of the effects of intraocular drug administration. Only one kind of self-assembling peptide may be used, or two or more kinds thereof may be used in combination.

Preferably, the self-assembling peptide to be used in the present invention is represented by the following amino acid sequence.

| | |
|---|---|
| Amino acid sequence: | a₁b₁c₁b₂a₂b₃db₄a₃b₅c₂b₆a₄ |

(In the amino acid sequence, a₁ to a₄ each represent a basic amino acid residue, b₁ to b₆ each represent a non-charged polar amino acid residue and/or a hydrophobic amino acid residue, provided that at least five amino acid residues out of the b₁ to b₆ are each a hydrophobic amino acid residue, c₁ and c₂ each represent an acidic amino acid residue, and d represents a hydrophobic amino acid residue.)

The self-assembling peptide represented by the above-mentioned amino acid sequence can serve as a synthetic vitreous material which is capable of maintaining its intraocular tamponade effect over a longer period of time. Further, the peptide represented by the above-mentioned amino acid sequence can form, under physiological conditions, a gel exhibiting excellent transparency and mechanical strength, and hence the peptide can be suitably used to produce a synthetic vitreous material.

L-amino acids or D-amino acids may be used as amino acids constituting the above-mentioned self-assembling peptide. Further, naturally-occurring amino acids or non-naturally-occurring amino acids may be used. Naturally-occurring amino acids are preferred because these amino acids are available at low prices and can be easily synthesized into a peptide.

In the amino acid sequence, a₁ to a₄ each represent a basic amino acid residue. Basic amino acids are preferably arginine, lysine, or histidine, more preferably arginine or lysine because these amino acids are highly basic. a₁ to a₄ may represent the same amino acid residue, or may represent amino acid residues different from each other.

In the amino acid sequence, b₁ to b₆ each represent a non-charged polar amino acid residue and/or a hydrophobic amino acid residue. Of those, at least five amino acid residues are hydrophobic amino acid residues. Hydrophobic amino acids are preferably alanine, leucine, isoleucine, valine, methionine, phenylalanine, tryptophan, glycine, or proline, and non-charged polar amino acids are preferably tyrosine, serine, threonine, asparagine, glutamine, or cysteine, because of the ready availability of these amino acids.

Preferably b₃ and b₄ are each independently any suitable hydrophobic amino acid residue, more preferably a leucine residue, an alanine residue, a valine residue, or an isoleucine residue, particularly preferably a leucine residue or an alanine residue. When the b₃ and b₄ respectively located at the sixth position and the eighth position are hydrophobic amino acid residues in the above-mentioned amino acid sequence, the three amino acid residues located at the sixth to eighth positions are consecutive hydrophobic amino acid residues. It is presumed that the hydrophobic region formed in the center of the amino acid sequence as mentioned above has a hydrophobic interaction or the like, thus being able to improve the strength of the resultant synthetic vitreous material, and hence the synthetic vitreous material can maintain its intraocular tamponade effect over a long term.

Preferably b₁ to b₆ are all hydrophobic amino acid residues, because the resultant self-assembling peptide can suitably form a β-sheet structure and can be self-assembled. More preferably b₁ to b₆ are each independently a leucine residue, an alanine residue, a valine residue, or an isoleucine residue, even more preferably a leucine residue or an alanine residue. In a preferred embodiment, 4 or more amino acid residues out of the b₁ to b₆ are leucine residues, particularly preferably, 5 or more amino acid residues out of the b₁ to b₆ are leucine residues, and most preferably, all amino acid residues are leucine residues. This is because the resultant self-assembling peptide has excellent solubility in water, thus easily enabling the preparation of a synthetic vitreous material, and because the strength of the synthetic vitreous material can be enhanced, and hence the synthetic vitreous material is capable of maintaining its intraocular tamponade effect over a long term.

In the above-mentioned amino acid sequence, c₁ and c₂ each represent an acidic amino acid residue. It is preferred that aspartic acid or glutamic acid is used as the acidic amino acids, because these amino acids are readily available. c₁ and c₂ may be the same amino acid residue or may be different amino acid residues.

In the above-mentioned amino acid sequenced represents a hydrophobic amino acid residue. Because d is a hydrophobic amino acid residue as mentioned above and the self-assembling peptide has a predetermined symmetric structure, it is reckoned that a synthetic vitreous material having a better mechanical strength is formed and the material can maintain an intraocular tamponade effect over a long term.

Preferably d is an alanine residue, a valine residue, a leucine residue, or an isoleucine residue. In this case, the length of the side chains of the amino acids located on the hydrophilic surface side in a β-sheet structure formed by the resulting self-assembling peptide can be non-complementary, but the self-assembling peptide can exhibit an excellent self-assembling ability, and a synthetic vitreous material that has better mechanical strength than conventional ones and is capable of maintaining an intraocular tamponade effect over a long term can be obtained.

The sum of the charges, in a neutral range of the amino acid residues contained in the above-mentioned self-assembling peptide, is substantially +2. That is, in the neutral range, plus charges and minus charges, which are derived from the side chains of the amino acid residues contained in the above-mentioned self-assembling peptide, are not offset. In addition, because both amino acid residues positioned at the N-terminal and C-terminal are basic amino acid residues, it is presumed that, for example, a static repulsive force as well as a static attractive force act between the molecules of the self-assembling peptide to be used in the present invention, a subtle balance is struck between the forces, thereby substantially preventing the occurrence of excessive assembly, and hence the self-assembling peptide can be formed into a stable gel without precipitating in the neutral range, which is close to physiological conditions. Note that, herein, the term “neutral range” refers to a range of pH 6 to 8, preferably pH 6.5 to 7.5.

The charge of the self-assembling peptide at each pH can be calculated in accordance with, for example, the method of Lehninger (Biochimie, 1979). The method of Lehninger can be performed with, for example, a program available on the web site of EMBL WWW Gateway to Isoelectric Point Service (http://www.embl-heidelberg.de/cgi/pi-wrapper.pl).

Preferred specific examples of the self-assembling peptide to be used in the present invention are shown below.

| | |
|---|---|
| n-RLDLRLALRLDLR-c | (SEQ ID NO: 1) |
| n-RLDLRLLLRLDLR-c | (SEQ ID NO: 2) |
| n-RADLRLALRLDLR-c | (SEQ ID NO: 3) |
| n-RLDLRLALRLDAR-c | (SEQ ID NO: 4) |
| n-RADLRLLLRLDLR-c | (SEQ ID NO: 5) |
| n-RADLRLLLRLDAR-c | (SEQ ID NO: 6) |
| n-RLDLRALLRLDLR-c | (SEQ ID NO: 7) |
| n-RLDLRLLARLDLR-c | (SEQ ID NO: 8) |

The above-mentioned self-assembling peptide can be produced by any suitable production method. Examples of the production method include a chemical synthetic method such as a solid-phase method, for example, an Fmoc method or a liquid-phase method, and a molecular biological method such as recombinant gene expression.

The above-mentioned self-assembling peptide may be a self-assembling peptide having any suitable modification (hereinafter referred to as a modified peptide). The modified peptide is a peptide which has a self-assembling ability and is prepared by subjecting the above-mentioned self-assembling peptide to any suitable modification to such an extent that the modified peptide is not toxic to the biological body, in particular, the ocular tissue. The site to which the modification is applied may be the N-terminal amino group of the above-mentioned self-assembling peptide or may be the C-terminal carboxyl group thereof. Alternatively, both the sites may be modified.

Any suitable modification can be selected as the above-mentioned modification as long as the resultant modified peptide has a self-assembling ability and is not toxic to the biological body, in particular, the ocular tissue. Examples of the modification include: the introduction of a protective group such as acetylation of an N-terminal or amidation of a C-terminal; the introduction of a functional group such as alkylation, esterification, or halogenation; hydrogenation; the introduction of a saccharide compound such as a monosaccharide, a disaccharide, an oligosaccharide, or a polysaccharide; the introduction of a lipid compound such as a fatty acid, a phospholipid, or a glycolipid; the introduction of an amino acid or a protein; the introduction of DNA; and the introduction of, for example, other compounds each having bioactivity. When an amino acid or a protein is introduced, the peptide obtained after the introduction is a peptide in which any suitable amino acid is added to the N-terminal and/or C-terminal of the above-mentioned self-assembling peptide. Herein, the added peptide is also included in the modified peptide. Only one kind of modification may be applied, or two or more kinds thereof may be applied in combination. For example, the following may be adopted: a desired amino acid is introduced into the C-terminal of the above-mentioned self-assembling peptide, yielding an added peptide, the N-terminal of the added peptide is acetylated, and the C-terminal thereof is amidated.

The above-mentioned added peptide (modified peptide) does not have, as a whole, features of the above-mentioned self-assembling peptide in some cases. Specific examples of such cases include the case that, owing to the addition of any suitable amino acid, the sequence toward the N-terminal direction and the sequence toward the C-terminal direction are asymmetric with respect to the hydrophobic amino acid residue at position 7 in the center, and the case that the added peptide has hydrophobic amino acids and hydrophilic amino acids in the same amounts. Even in such case, because the above-mentioned self-assembling peptide has an extremely excellent self-assembling ability, the added peptide obtained by adding any appropriate amino acid to the self-assembling peptide can also provide a synthetic vitreous material which has excellent mechanical strength and is capable of maintaining an intraocular tamponade effect over a long term.

In the case of introducing some amino acids or proteins, the number of the amino acid residues constituting the modified peptide obtained after the introduction is preferably 14 to 200, more preferably 14 to 100, even more preferably 14 to 50, particularly preferably 14 to 30, most preferably 14 to 20. When the number of the amino acid residues is more than 200, the self-assembling ability of the above-mentioned self-assembling peptide is sometimes impaired.

The kinds and positions of the amino acids to be introduced can be properly set depending on the applications of the resultant modified peptide and the like. It is preferred that amino acids are introduced so that a hydrophobic amino acid and a hydrophilic amino acid are positioned alternately, starting from the arginine residues/residue (hydrophilic amino acids/acid) located at the N-terminal and/or the C-terminal of the above-mentioned self-assembling peptide.

The above-mentioned modification can be performed by any suitable method depending on the kind thereof and the like.

C. Additive(s)

The synthetic vitreous material of the present invention may include any suitable additive(s) in addition to the above-mentioned self-assembling peptide and salt. Examples of the additive(s) include any suitable chemicals, for example, low-molecular-weight compounds, nucleic acids such as DNA and RNA, and antibodies such as Lucentis, Avastin, and Macugen.

D. Production Method for the Synthetic Vitreous Material

The synthetic vitreous material of the present invention can be producedby any suitable method. For example, the above-mentioned self-assembling peptide is dissolved in distilled water so as to prepare an aqueous peptide solution having a desired concentration, and the aqueous peptide solution, the above-mentioned salt, any suitable additive, if required, and a solvent are agitated and mixed by using any suitable agitation means, thereby being able to yield a synthetic vitreous material. Another example of a suitable method is as follows: the above-mentioned aqueous peptide solution, the above-mentioned salt solution, and any suitable additive(s), if required, are agitated and mixed by using any suitable agitation means, thereby being able to yield a synthetic vitreous material.

E. Method of Use for the Synthetic Vitreous Material

The synthetic vitreous material of the present invention can be injected into an eyeball by using any suitable means. For example, the synthetic vitreous material of the present invention is filled in a syringe, followed by sterilization treatment, and the synthetic vitreous material can be injected into an eyeball by using a syringe. The synthetic vitreous material of the present invention has excellent handleability, and hence it is possible to easily inject the synthetic vitreous material into an eyeball even when an injection needle is used that is thinner than a 25-gauge injection needle usually used for injection into an eyeball.

### EXAMPLES

Hereinafter, the present invention is specifically described on the basis of examples, but the present invention is not limited by these examples. Note that the osmotic pressure of each synthetic vitreous material was measured as described below.

### (Measurement of Osmotic Pressure)

Each synthetic vitreous material was diluted by using distilled water (trade name: Official Otsuka Distilled Water, manufactured by Otsuka Pharmaceutical Factory, Inc.) until a solution state was achieved. Next, the osmotic pressure of each diluted synthetic vitreous material was measured by using an osmometer (trade name: Osmometer 3900, manufactured by Advanced Instruments, Inc.) in accordance with the osmometry (osmolality measurement method) described in the Pharmacopeia of Japan. The value of each osmotic pressure obtained was proportionally calculated on the basis of each dilution rate, thereby determining the osmotic pressure of each synthetic vitreous material.

### Example 1

A self-assembling peptide (trade name: PanaceaGel SPG-178, manufactured by Menicon Co., Ltd., 1 w/v %) was mixed with distilled water (trade name: Official Otsuka Distilled Water, manufactured by Otsuka Pharmaceutical Factory, Inc.), yielding a peptide aqueous solution having a peptide concentration of 0.15 w/v %. The thus-yielded peptideaqueous solution and a salt solution 1 (a diluent for an oxiglutatione solution with an osmotic pressure of 308 mOsm/kg packed in Opeaqua (trademark), manufactured by Showa Yakuhin Kako Co., Ltd.) were mixed at a volume ratio of 2:1, yielding a synthetic vitreous material 1. Table 1 shows the concentration of the self-assembling peptide, the ratio of the salt solution, and the osmotic pressure in the obtained synthetic vitreous material.

### Example 2

A synthetic vitreous material 2 was obtained in the same manner as that in Example 1, except that a salt solution 2 (a diluent for an oxiglutatione solution with an osmotic pressure of 308 mOsm/kg packed in BSS PLUS (trademark), which is a trade name and is manufactured by ALCON JAPAN LTD.) was used in place of the salt solution 1. Table 1 shows the concentration of the self-assembling peptide, the ratio of the salt solution, and the osmotic pressure in the obtained synthetic vitreous material.

### Example 3

A synthetic vitreous material 3 was obtained in the same manner as that in Example 2, except that the peptide concentration of the resultant peptide aqueous solution was set to 0.45 w/v %. Table 1 shows the concentration of the self-assembling peptide, the ratio of the salt solution, and the osmotic pressure in the obtained synthetic vitreous material.

### Example 4

A synthetic vitreous material 4 was obtained in the same manner as that in Example 1, except that the peptide concentration of the resultant peptide aqueous solution was set to 0.075 w/v %. Table 1 shows the concentration of the self-assembling peptide, the ratio of the salt solution, and the osmotic pressure in the obtained synthetic vitreous material.

### Example 5

A synthetic vitreous material 5 was obtained in the same manner as that in Example 2, except that the peptide concentration of the resultant peptide aqueous solution was set to 0.25 w/v % and the mixing ratio of the peptide aqueous solution to the salt solution 2 was set to a volume ratio of 2:3. Table 1 shows the concentration of the self-assembling peptide, the ratio of the salt solution, and the osmotic pressure in the obtained synthetic vitreous material.

### Comparative Example 1

A peptide aqueous solution having a peptide concentration of 1 w/v % was prepared in the same manner as that in Example 1. Distilled water (trade name: Official Otsuka Distilled Water, manufactured by Otsuka Pharmaceutical Factory, Inc.) was further added to the peptide aqueous solution so that the concentration of the self-assembling peptide came down to 0.1 w/v %, yielding a synthetic vitreous material C1. Table 1 shows the concentration of the self-assembling peptide, the ratio of the salt solution, and the osmotic pressure in the obtained synthetic vitreous material.

### Comparative Example 2

A synthetic vitreous material C2 was obtained in the same manner as that in Example 1, except that the peptide concentration of the resultant peptide aqueous solution was set to 1 w/v % and the mixing ratio of the peptide aqueous solution to the salt solution was set to a volume ratio of 3:7. Table 1 shows the concentration of the self-assembling peptide, the ratio of the salt solution, and the osmotic pressure in the obtained synthetic vitreous material.

### Comparative Example 3

A synthetic vitreous material C3 was obtained in the same manner as that in Example 1, except that the peptide concentration of the resultant peptide aqueous solution was set to 0.2 w/v % and the mixing ratio of the peptide aqueous solution to the salt solution was set to a volume ratio of 9:1. Table 1 shows the concentration of the self-assembling peptide, the ratio of the salt solution, and the osmotic pressure in the obtained synthetic vitreous material.

TABLE 1

| | Peptide | | | | |
|---|---|---|---|---|---|
| | concentration | | | | |
| | in | | Synthetic vitreous material | | |
| | self- | | Concentration | | |
| | assembling | | of | | |
| | peptide | | self- | Ratio of | Osmotic |
| | aqueous | | assembling | salt | pressure |
| | solution | Salt | peptide | solution | (mOsm/ |
| | (w/v %) | solution | (w/v %) | (v/v %) | kg) |
| Example 1 | 0.15 | Salt | 0.1 | 33 | 103 |
| | | solution | | | |
| | | 1 | | | |
| Example 2 | 0.15 | Salt | 0.1 | 33 | 103 |
| | | solution | | | |
| | | 2 | | | |
| Example 3 | 0.45 | Salt | 0.3 | 33 | 103 |
| | | solution | | | |
| | | 2 | | | |
| Example 4 | 0.075 | Salt | 0.05 | 33 | 103 |
| | | solution | | | |
| | | 1 | | | |
| Example 5 | 0.25 | Salt | 0.1 | 60 | 185 |
| | | solution | | | |
| | | 2 | | | |
| Comparative | 1 | — | 0.1 | — | 0 |
| Example 1 | | | | | |
| Comparative | 1 | Salt | 0.3 | 70 | 216 |
| Example 2 | | solution | | | |
| | | 1 | | | |
| Comparative | 0.2 | Salt | 0.18 | 10 | 31 |
| Example 3 | | solution | | | |
| | | 1 | | | |

(Evaluation)

Each of the synthetic vitreous materials obtained in Examples 1 to 5 and Comparative Examples 1 to 3 was heated to 37° C. by using an incubator (trade name: CO₂ Incubator, manufactured by SANYO Electric Co., Ltd.), and the following evaluations were performed.

### <Tamponade Effect, Physical Properties, and Transparency>

The tamponade effect, physical properties, and transparency of each of the heated synthetic vitreous materials were visually confirmed and evaluated. The tamponade effect was evaluated as described below. Table 2 shows the results of the evaluation.

Tamponade Effect
⊚: Having a high tamponade effect.
o: Having a tamponade effect.
x: Having no tamponade effect.

### <Handleability>

Each of the heated synthetic vitreous materials was filled in an injector (injection needle: 26 gauge), and the handleability thereof was evaluated on the basis of the feeling (handling) sensed at the time of ejecting each of the materials. Table 2 shows the results of the evaluation.

TABLE 2

| | | Property and state | | |
|---|---|---|---|---|
| | | of synthetic | | |
| | Tamponade | vitreous | | Handle- |
| | effect | material | Transparency | ability |
| Example 1 | ◯ | State of a gel | ◯ | Easy |
| | | having high | | |
| | | fluidity | | |
| Example 2 | ◯ | State of a gel | ◯ | Easy |
| | | having high | | |
| | | fluidity | | |
| Example 3 | ⊚ | State of a gel | ◯ | Easy |
| | | having moderate | | |
| | | fluidity | | |
| Example 4 | ◯ | State of a gel | ◯ | Easy |
| | | having high | | |
| | | fluidity | | |
| Example 5 | ◯ | State of a fragile | ◯ | Easy |
| | | agglomerate | | |
| | | having low | | |
| | | fluidity | | |
| Comparative | X | Liquid state | ◯ | Easy |
| Example 1 | | | | |
| Comparative | ⊚ | State of a gel | X | Easy |
| Example 2 | | having moderate | | |
| | | fluidity | | |
| Comparative | X | Liquid state | ◯ | Easy |
| Example 3 | | | | |

Each of the synthetic vitreous materials obtained in Examples 1 to 5 had a tamponade effect, excellent handleability, and high transparency, and hence the materials can be suitably used as synthetic vitreous materials. A 25-gauge injection needle is usually used for injection into an eyeball. The synthetic vitreous material of the present invention had excellent handleability even when a 26-gauge injection needle that is thinner than the usual one was used.

The synthetic vitreous materials obtained in Comparative Example 1 and Comparative Example 3 were in liquid states, and hence no satisfactory tamponade effect was exhibited. On the other hand, the synthetic vitreous material obtained in Comparative Example 2 had an excellent tamponade effect and handleability but an inferior transparency, and hence the material was not suitable for being used as a synthetic vitreous material.

### Test Example

Injection Test into Eyeball of Domestic Rabbit

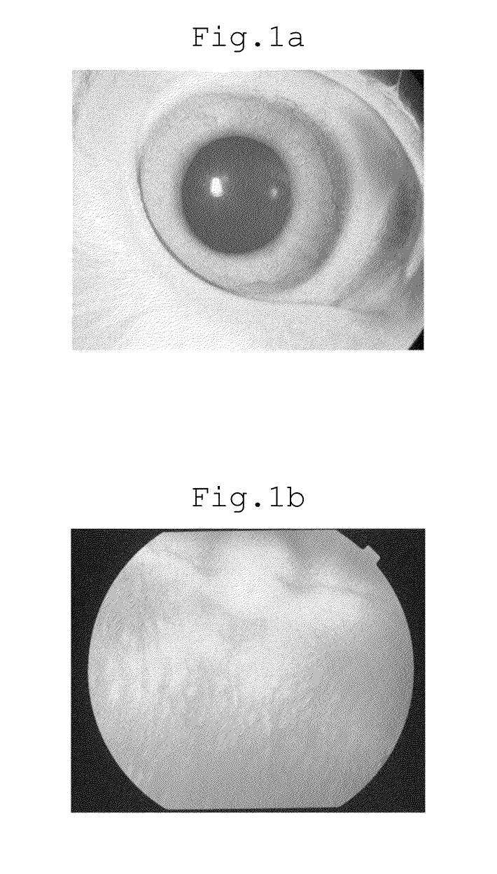

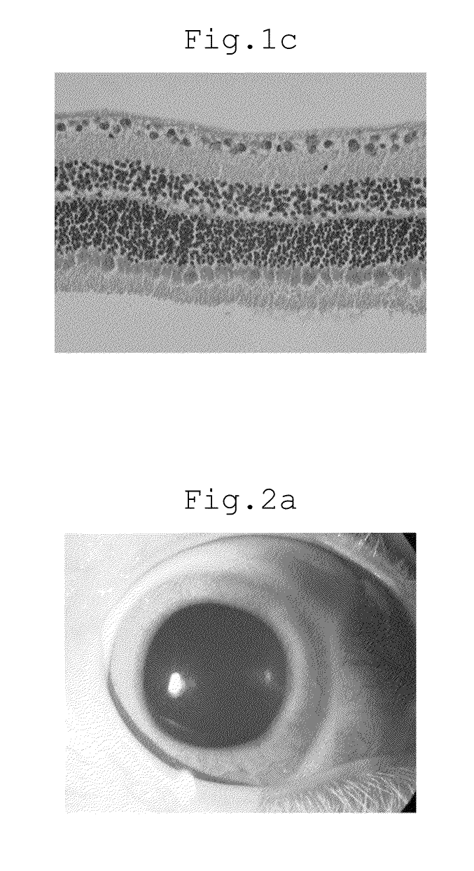

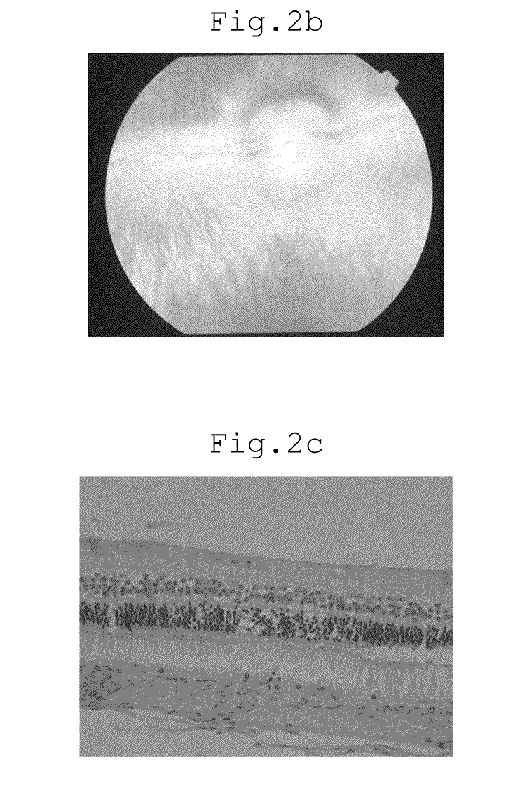

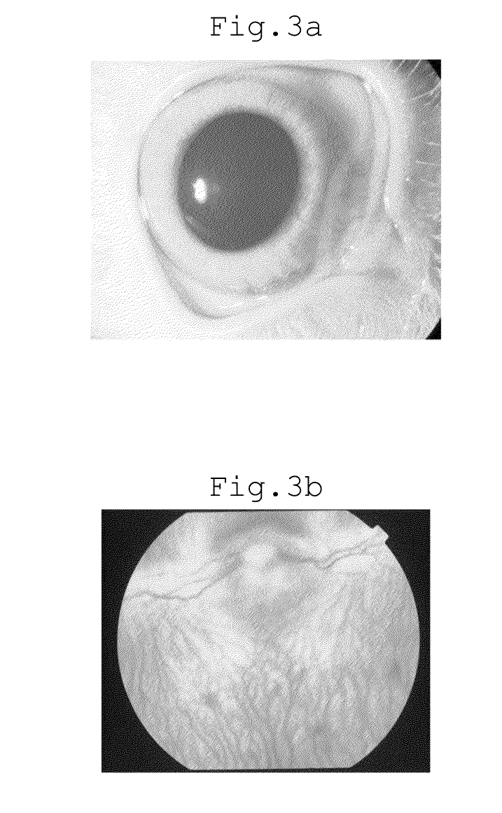

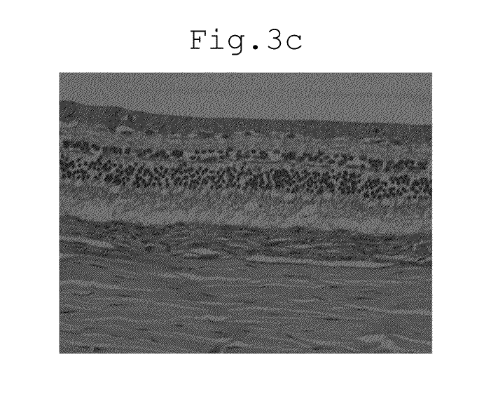

21 white domestic rabbits each having a body weight of 2 kg were used as specimens, and ketamine and xylazine were intramuscularly injected thereinto at 15 mg/kg and 10 mg/kg, respectively, thereby deeply anesthetizing the rabbits. Disappearance of corneal reflex and disappearance of reaction to pain stimulation were confirmed. Subsequently, vitrectomy was performed on one eyeball of each specimen, and then the synthetic vitreous material 2 obtained in Example 2 was injected into each eyeball to complete the operation. A three-port system (25 G), which has been widely used in human clinical medicine, was used as the method for the operation. One day after the operation, three days later, one week later, two weeks later, three weeks later, one month later, and three months later, each eyeball into which the synthetic vitreous material had been injected was observed with a slit-lamp microscope and a fundus microscope, followed by the measurement of electroretinograms. On each observation day, each eyeball into which the synthetic vitreous material had been injected was extirpated from three domestic rabbit specimens, followed by HE staining, and the state of each retina was observed. FIG. 1a shows a photograph of an anterior eye segment, FIG. 1b shows a photograph of an ocular fundus, and FIG. 1c shows a photograph of an HE-stained retinal tissue, the photographs being taken one week after the operation. Similarly, FIG. 2a shows a photograph of an anterior eye segment, FIG. 2b shows a photograph of an ocular fundus, and FIG. 2c shows a photograph of an HE-stained retinal tissue, the photographs being taken one month after the operation. FIG. 3a shows a photograph of an anterior eye segment, FIG. 3b shows a photograph of an ocular fundus, and FIG. 3c shows a photograph of an HE-stained retinal tissue, the photographs being taken three months after the operation.

On all observation days, all crystalline lenses and all synthetic vitreous materials were not cloudy, and all ocular fundi were able to be observed. Even one month after the operation, the onset of a cataract was not observed and the synthetic vitreous material was found to be not toxic to the retinal tissue. In addition, even one month after the operation, the synthetic vitreous material remained in each eyeball and a good tamponade effect thereof was maintained. Further, the synthetic vitreous material itself remained without hardening and becoming cloudy. Even three months after the operation, the onset of a cataract was not observed and the synthetic vitreous material was found to be not toxic to the retinal tissue. In addition, the synthetic vitreous material neither hardened nor became cloudy and maintained its good tamponade effect. As mentioned above, the synthetic vitreous material of the present invention had excellent handleability, and even after as long a period of time as three months had passed, the synthetic vitreous material was not toxic to the ocular tissue and was able to maintain its good intraocular tamponade effect.

### INDUSTRIAL APPLICABILITY

The synthetic vitreous material of the present invention can be suitably used as a tamponade material which is used after vitrectomy.

### Sequence Listing Free Text

SEQ ID NO. 1 is a self-assembling peptide to be used in the present invention.

SEQ ID NO. 2 is a self-assembling peptide to be used in the present invention.

SEQ ID NO. 3 is a self-assembling peptide to be used in the present invention.

SEQ ID NO. 4 is a self-assembling peptide to be used in the present invention.

SEQ ID NO. 5 is a self-assembling peptide to be used in the present invention.

SEQ ID NO. 6 is a self-assembling peptide to be used in the present invention.

SEQ ID NO. 7 is a self-assembling peptide to be used in the present invention.

SEQ ID NO. 8 is a self-assembling peptide to be used in the present invention.

1. A synthetic vitreous material, comprising:
a self-assembling peptide and
a salt,
wherein the synthetic vitreous material has an osmotic pressure of 40 mOsm/kg to 200 mOsm/kg.

2. The synthetic vitreous material according to claim 1, wherein the synthetic vitreous material comprises the self-assembling peptide at 0.01 w/v % to 0.5 w/v %.

3. The synthetic vitreous material according to claim 1, wherein the self-assembling peptide has the following amino acid sequence:
a₁b₁c₁b₂a₂b₃db₄a₃b₅c₂b₆a₄
where:
a₁ to a₄ are each a basic amino acid residue,
b₁ to b₆ are each an uncharged polar amino acid residue and/or a hydrophobic amino acid residue, provided that at least five amino acid residues out of b₁ to b₆ are hydrophobic amino acid residues,
c₁ and c₂ are each an acidic amino acid residue, and
d is a hydrophobic amino acid residue.

4. The synthetic vitreous material according to claim 3, wherein b₁ to b₆ in the amino acid sequence are each independently an alanine residue, a valine residue, a leucine residue, or an isoleucine residue.

5. The synthetic vitreous material according to claim 3, wherein d in the amino acid sequence is an alanine residue, a valine residue, a leucine residue, or an isoleucine residue.

6. The synthetic vitreous material according to claim 3, wherein:
a₁ to a₄ are each independently an arginine residue, a lysine residue or a histidine reside,
b₁ to b₆ are each independently a tyrosine residue, a serine residue, a threonine residue, an asparagine residue, a glutamine residue, a cysteine residue, an alanine residue, a leucine residue, an isoleucine residue, a valine residue, a methionine residue, a phenylalanine residue, a tryptophan residue, a glycine residue, or a proline residue, provided that at least five amino acid residues out of b₁ to b₆ are hydrophobic amino acid residues,
c₁ and c₂ are each independently an aspartic residue or a glutamic residue, and
d is an alanine residue, a leucine residue, an isoleucine residue, a valine residue, a methionine residue, a phenylalanine residue, a tryptophan residue, a glycine residue, or a proline residue.

7. The synthetic vitreous material according to claim 1, wherein the synthetic vitreous material is a viscoelastic substance at room temperature.

8. The synthetic vitreous material according to claim 1, wherein the self-assembling peptide and the salt are dissolved in an aqueous solution.

9. The synthetic vitreous material according to claim 8, wherein the aqueous solution comprises a physiological saline solution, a Ringer's solution, or an intraocular irrigating solution.

10. The synthetic vitreous material according to claim 1, wherein the synthetic vitreous material comprises the self-assembling peptide at 0.05 w/v % to 0.4 w/v %.

11. The synthetic vitreous material according to claim 1, wherein the self-assembling peptide is selected from the group consisting of:
n-RLDLRLALRLDLR-c,
n-RLDLRLLLRLDLR-c,
n-RADLRLALRLDLR-c,
n-RLDLRLALRLDAR-c,
n-RADLRLLLRLDLR-c,
n-RADLRLLLRLDAR-c,
n-RLDLRALLRLDLR-c,
and
n-RLDLRLLARLDLR-c,
wherein:
n is the N-terminal,
c is the C-terminal,
R is arginine,
L is leucine,
D is aspartic acid, and
A is alanine.

12. The synthetic vitreous material according to claim 4, wherein d in the amino acid sequence is an alanine residue, a valine residue, a leucine residue, or an isoleucine residue.

13. The synthetic vitreous material according to claim 12, wherein:
a₁ to a₄ are each independently an arginine residue, a lysine residue or a histidine reside,
b₁ to b₆ are all hydrophobic amino acid residues, and
c₁ and c₂ are each independently an aspartic residue or a glutamic residue.

14. The synthetic vitreous material according to claim 13, wherein b₁ to b₆ are each leucine residues.

15. The synthetic vitreous material according to claim 10, wherein the self-assembling peptide is selected from the group consisting of:
n-RLDLRLALRLDLR-c,
n-RLDLRLLLRLDLR-c,
n-RADLRLALRLDLR-c,
n-RLDLRLALRLDAR-c,
n-RADLRLLLRLDLR-c,
n-RADLRLLLRLDAR-c,
n-RLDLRALLRLDLR-c,
and
n-RLDLRLLARLDLR-c,
wherein:
n is the N-terminal,
c is the C-terminal,
R is arginine,
L is leucine,
D is aspartic acid, and
A is alanine.

16. The synthetic vitreous material according to claim 15, wherein the synthetic vitreous material is a viscoelastic substance at room temperature.

17. The synthetic vitreous material according to claim 16, wherein the self-assembling peptide and the salt are dissolved in an aqueous solution.

18. A method of treating a patient in need thereof, comprising:
injecting a therapeutically effective amount of the synthetic vitreous material of claim 17 into an eye of the patient.

19. A method of treating a patient in need thereof, comprising:
injecting a therapeutically effective amount of the synthetic vitreous material of claim 1 into an eye of the patient.


SEQUENCE LISTING (8 sequences)

SEQ ID NO: 1 - protein, 13 aa, Artificial
  self-assembling peptide
RLDLRLALRLDLR

SEQ ID NO: 2 - protein, 13 aa, Artificial
  self-assembling peptide
RLDLRLLLRLDLR

SEQ ID NO: 3 - protein, 13 aa, Artificial
  self-assembling peptide
RADLRLALRLDLR

SEQ ID NO: 4 - protein, 13 aa, Artificial
  self-assembling peptide
RLDLRLALRLDAR

SEQ ID NO: 5 - protein, 13 aa, Artificial
  self-assembling peptide
RADLRLLLRLDLR

SEQ ID NO: 6 - protein, 13 aa, Artificial
  self-assembling peptide
RADLRLLLRLDAR

SEQ ID NO: 7 - protein, 13 aa, Artificial
  self-assembling peptide
RLDLRALLRLDLR

SEQ ID NO: 8 - protein, 13 aa, Artificial
  self-assembling peptide
RLDLRLLARLDLR